(19) United States
(12) Patent Application Publication
(10) Pub. No.: US 2013/0156871 A1
(43) Pub. Date: Jun. 20, 2013

(54) Nasal Wash Solution

(71) Applicant: Water Pik, Inc., Fort Collins, CO (US)

(72) Inventor: Ted Keller, Centennial, CO (US)

(73) Assignee: WATER PIK, INC., Fort Collins, CO (US)

(21) Appl. No.: 13/722,748

(22) Filed: Dec. 20, 2012

Related U.S. Application Data
(60) Provisional application No. 61/577,945, filed on Dec. 20, 2011.

(51) Int. Cl. A61K 9/00

(52) U.S. Cl. CPC A61K 9/0043
USPC 424/742; 514/769; 514/784; 424/744; 514/729

(57) ABSTRACT

A composition provides an isotonic nasal irrigation solution with pH less than about 7.0. The composition may be formed of a salt and a pH buffer and may include an additive. The composition may be mixed with water to form the solution. The composition may aid in the treatment of various nasal or sinus disorders such as, for example, dryness, rhinitis, and sinusitis. The isotonic irrigation solution may also aid in the removal of contaminants from the sinus and nasal cavities such as blood, mucus, dirt, pollen, allergens, viruses, and bacterial. The composition may be liquid or dry.

### CROSS-REFERENCE TO RELATED APPLICATIONS

The present application claims the benefit of priority pursuant to 35 U.S.C. §119(e) of U.S. Provisional Application No. 61/577,945, filed Dec. 20, 2011, and entitled “Nasal Wash Solution,” which application is incorporated herein by reference in its entirety.

### BACKGROUND

The subject technology relates generally to nasal and sinus hygiene, and more particularly to a solution for nasal and sinus wash.

The terms “nasal irrigation,” “nasal lavage,” “sinus wash,” and “nasal wash” generally refer to the introduction of saline into the nasal and sinus cavities for the purpose of cleansing or medicating the sinus/nasal cavities and membranes. This procedure has become more commonly accepted in daily hygiene routines as the health benefits associated with its practice become more widely known.

Studies show that nasal irrigation may help ameliorate, lessen the duration, or even prevent episodic, perennial, and chronic sinus conditions including, for example, dryness, rhinitis, and sinusitis. Studies have also demonstrated the benefits of nasal irrigation in speeding recovery from nasal and sinus surgery.

Nasal/sinus washing may be performed to moisturize, medicate, clean, or rinse the nasal and sinus passages and cavities. Irrigation may be performed manually with simple gravity or by manual pressurization of the saline solution through use of a squeeze bottle. Additionally, modified electric irrigators have also been used in sinus washing. One study suggested that modification of electric oral irrigators generating a pulsating stream of nasal irrigation solution may remove bacteria better relative to other methods.

Solutions for use in nasal/sinus wash are generally isotonic solutions of saline and buffering agents to maintain the solution at a basic pH. Bicarbonate, due in part to its major role in mammalian physiology and favorable buffering characteristics, is often used as a buffering agent in nasal wash solutions. Bicarbonate is a major constituent of blood and helps to maintain a basic pH of about 7.4. A blood pH of less than about 6.8 may lead to cell damage or even death. In the sinuses, the pH may vary between about 5 to about 8, but is generally about 6.4. However, as stated above, nasal irrigation tends to be performed with solutions having a basic pH.

### SUMMARY

One embodiment of the present nasal/sinus wash composition comprises a salt and a pH buffer, wherein when mixed with water the composition may provide an isotonic saline solution of pH below about 7.0. Preferably, the pH of the nasal wash solution may be about 6.7. In some embodiments, the salts may comprise sodium chloride, sodium bicarbonate, and sodium ascorbate, and the buffer may further comprise sodium bicarbonate, sodium ascorbate, or both.

In various embodiments, the composition is intended to be mixed with water prior to use. The composition may be provided in dry or liquid form. The composition may further be provided in bulk to allow a user to measure out the desired amount of composition, or the composition may be provided in individually-packaged, pre-measured amounts, or doses.

The water used to make the isotonic wash solution may be treated, for example, by distillation, de-ionization, filtration, or a combination thereof. The water may be sterile and may be obtained from a bottled source or from a home tap.

In various embodiments the water may be sterilized, or prepared by boiling the water and cooling or allowing the water to cool prior to use in order to aid in sterilization. In various embodiments the water may be cooled to approximately body temperature or lower.

Hypertonic, and hypotonic solutions are also disclosed. For example, by mixing the composition with less or more than the appropriate volume of water, or by adding more or less composition to the appropriate volume of water, or a combination thereof, the user may choose to create an hypertonic or hypotonic solution of the same or similar pH.

Various embodiments of the present composition may include additives. Additives may be, for example, moisturizers, flavorants, oils, herbs, and medicaments. These additives may aid in the treatment of a sinus condition, inhibit or prevent bacterial growth, or increase the pleasurability of the nasal/sinus wash experience.

Additives, may also aid in changing the sinus environment by, for example, increasing mucus production, thinning mucus, changing the pH of the mucus, stimulating transport of the mucus from the nasal and sinus cavities, or shrinking the nasal mucosal epithelium.

Methods of using the present composition in the treatment of sinus or nasal conditions are also provided. For example, hypertonic solutions may be used to treat sinusitis in a patient in need of such treatment.

### BRIEF DESCRIPTION OF THE DRAWING

FIG. 1 is a graph showing the survival of S. epidermidis exposed to various sinus irrigation solutions for various times.

### DETAILED DESCRIPTION

One embodiment of the present nasal/sinus wash compositions comprises a salt and a pH buffer, wherein when mixed with water the composition may provide an isotonic saline solution of pH below about 7.0. In some cases the pH of the solution may be less than about 7.0, 6.9, 6.8, 6.7, 6.6, or 6.5 and greater than about 6.4, 6.5, 6.6, 6.7, 6.8, or 6.9.

Preferably, the pH of the nasal wash solution may be about 6.7. In some embodiments, the salts may comprise sodium chloride, sodium bicarbonate, and sodium ascorbate, and the buffer may further comprise sodium bicarbonate, sodium ascorbate, or both.

In various embodiments, the composition is intended to be mixed with water prior to use. The water used may be generally body temperature, that is around 97 degrees Fahrenheit or room temperature, that is between about 65 and about 85 degrees Fahrenheit. Body temperature may be about 37° C. and room temperature may be about 23° C.

In some cases, the solution may be used at about 37° C. In some cases, the solution temperature may be greater than about 25° C., 30° C., 34° C., 35° C., 36° C., 37° C., 38° C., or 39° C., and lower than about 40° C., 39° C., 38° C., 37° C., 36° C., 35° C., 34° C., 33° C., 32° C., 31° C., 30° C., or 29° C.

The composition may be provided in liquid or dry form, such as a powder or granular form. The composition may further be provided in bulk to allow a user to measure out the desired amount of composition, or the composition may be provided in individually-packaged, pre-measured amounts or doses.

The water used to make the isotonic wash solution may be treated, for example, by distillation, de-ionization, filtration, or a combination thereof. The water is sterile and may be from a bottled source or may be obtained from a home tap. In some cases, the water may be sterilized by boiling prior to mixing with a solution. In these cases, the solution may be cooled prior to use. Hypertonic, and hypotonic solutions are also possible by mixing the composition with less or more than the appropriate volume of water, or by adding more or less composition to the appropriate volume of water, or a combination thereof.

Various embodiments of the present composition may include additives. Additives may be, for example, moisturizers, flavorants, oils, herbs, and medicaments. These additives may aid in the treatment of a sinus condition, inhibit or prevent bacterial growth, or increase the pleasurability of the experience. Additives, may also aid in changing the sinus environment by, for example, increasing mucus production, thinning mucus, changing the pH of the mucus, stimulating transport of the mucus from the nasal and sinus cavities, or shrinking the nasal mucosal epithelium.

Methods of using the present composition in the treatment of sinus or nasal conditions are also provided. For example, hypertonic solutions may be used to treat sinusitis in a patient in need of such treatment.

In various embodiments, the described composition may be used to control, reduce, or inhibit the growth or the number of a variety of organisms. In some embodiments the organism may be selected from one or more strain of Staphylococcus and Haemophilus. In some embodiment the one or more organisms may be selected from Staphylococcus epidermidis, Staphylococcus aureus, Staphylococcus pneumoniae, and Haemophilus influenzae. Growth is controlled, reduced, or inhibited where, when the organism is grown in the presence of the composition, a culture of the organism has progressively fewer viable organisms over time. For example, successively removed aliquots of a culture of organisms grown, at appropriate conditions (shaking, temperature, humidity, etc), in a solution of the presently described composition will contain fewer CFUs (colony forming units, or single cells) over time.

In one embodiment, a dry formulation may be 80% to 88% sodium chloride, 9% to 20% sodium ascorbate, and 0% to 2% sodium bicarbonate by weight. In some embodiments, the sodium ascorbate concentration may be greater than about 8%, 9%, 10%, 11%, 12%, 13%, 14%, 15%, 16%, 17%, 18%, 19%, or 20%, and less than about 21%, 20%, 19%, 18%, 17%, 16%, 15%, 14%, 13%, 12%, 11%, 10%, or 9% by weight. In some cases, the sodium chloride concentration may be greater than about 75%, 76%, 77%, 78%, 79%, 80%, 81%, 82%, 83%, 84%, 85%, 86%, 87%, 88%, 89%, or 90%, and less than about 91%, 90%, 89%, 88%, 87%, 86%, 85%, 84%, 83%, 82%, 81%, 80%, 79%, 78%, 77%, or 76% by weight. In some case, the sodium bicarbonate concentration may be greater than about 0%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, or 2.1% and less than about 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% by weight. For embodiments with additives, the additives will generally account for less than 0.5% of the total weight of the formulation.

In some cases, 1 gram of composition may comprise between 0.790 and 0.890 grams of sodium chloride, between 0.07 and 0.21 grams of sodium ascorbate, and 0.000 and 0.030 grams of sodium bicarbonate. In various embodiments, 1 gram of the composition may comprise greater than about 0.800, 0.805, 0.810, 0.815, 0.820, 0.825, 0.830, 0.835, 0.840, 0.845, 0.850, 0.855, 0.860, 0.865, 0.870, or 0.875 and less than about 0.880, 0.875, 0.870, 0.865, 0.860, 0.855, 0.850, 0.845, 0.840, 0.835, 0.830, 0.825, 0.820, 0.815, 0.810, or 0.805 grams of sodium chloride; and greater than about 0.090, 0.095, 0.100, 0.105, 0.110, 0.115, 0.120, 0.125, 0.130, 0.135, 0.140, 0.145, 0.150, 0.155, 0.160, 0.165, 0.170, 0.175, 0.180, 0.185, 0.190, or 0.195 and less than about 0.205, 0.200, 0.195, 0.190, 0.185, 0.180, 0.175, 0.170, 0.165, 0.160, 0.155, 0.150, 0.145, 0.140, 0.135, 0.130, 0.125, 0.120, 0.115, 0.100, or 0.095 grams of sodium ascorbate; and greater than about 0.000, 0.005, 0.010, or 0.015 and less than about 0.020, 0.015, 0.010, or 0.005 grams of sodium bicarbonate. In some cases, 1 gram of the composition may comprise greater than about 0.000, 0.005, 0.010, 0.015, 0.020, 0.025, 0.030, 0.035, 0.040, or 0.045 and less than about 0.050, 0.045, 0.040, 0.035, 0.030, 0.025, 0.020, 0.015, 0.010, or 0.050 grams of an additive.

In various embodiments other buffers may be used to achieve the desired results. For example, in some embodiments sodium phosphate dibasic, Na₂HPO₄, may be mixed with sodium phosphate monobasic, NaH₂PO₄, to prepare a composition that when mixed with water may result in a pH around 6.7. In other embodiments, sodium phosphate monobasic and/or dibasic may be mixed with sodium bicarbonate and/or sodium ascorbate to create a composition that may result in a solution of the desired pH.

In some embodiments of the current composition, the dry formulation may be provided in individually packaged, pre-measured doses of between about 2 and about 3 grams, and when dissolved in about 230 to about 250 milliliters of water, an isotonic solution with pH 6.7 may be created. In some cases, the weight of one milliliter of water is about 1 gram. In various embodiments the ratio of composition to water in grams to moles is about 1:100. In some cases, the ratio may be between about 1:80 to 1:120. In some cases the ratio may be greater than about 1:80, 1:85, 1:90, 1:91, 1:92, 1:93, 1:94, 1:95, 1:96, 1:97, 1:98, 1:99, 1:100, 1:101, 1:102, 1:103, 1:104, 1:105, 1:106, 1:107, 1:108, 1:109, 1:110, 1:111, 1:112, 1:113, 1:114, 1:115, 1:116, 1:117, 1:118, 1:119, 1:120, 1:125, and 1:130 and less than about 1:130, 1:125, 1:120, 1:119, 1:118, 1:117, 1:116, 1:115, 1:114, 1:113, 1:112, 1:111, 1:110, 1:109, 1:108, 1:107, 1:106, 1:105, 1:104, 1:103, 1:102, 1:101, 1:100, 1:99, 1:98, 1:97, 1:96, 1:95, 1:94, 1:93, 1:92, 1:91, 1:90, and 1:85.

In various embodiments, the solution may be approximately body temperature, that is about 97 degrees Fahrenheit. In other embodiments the solution may be generally room temperature, that is between about 65 degrees and about 85 degrees Fahrenheit. In other embodiments the solution may be selected by the user to maximize individual needs and comfort.

A pre-measured amount may be referred to as a dose. The appropriate amount of water for creating an isotonic solution from a pre-measured dose may vary depending on the application, sinus condition, method of delivery, and or preferences of the user or patient. A specific dose may be designed to be dissolved in, mixed with, or added to a specific volume of water in order to produce an isotonic solution. When a hypertonic or hypotonic solution is desired, either less or more water than specified will be used respectively. For example, a 2 gram dose of the current composition may call for a 200 ml volume of water, while two doses of the composition in the same volume of water may provide a hypertonic solution. Alternatively, a 100 ml solution containing 2 grams of the current composition may also be hypertonic.

In addition to pre-measured, individually packaged dose amounts of the current composition, the composition may also be provided in bulk, for example, a container that holds many, unpackaged doses. When a bulk composition is provided, the user may have greater freedom to measure out a desired dose. In some embodiments, the bulk composition may be provided with a measuring device such as a dropper for liquid bulk compositions, or a spoon or scoop for dry formulations.

### Additives

Additives may be included in the current formulation. Additives may help to treat a condition, reduce congestion, or relieve or lessen inflammation. Additives may further treat a sinus condition, or inhibit or prevent bacterial growth. Additives may also aid in increasing the pleasurability of the experience.

Additives may include, for example, flavorings, oils, herbal extracts, medicaments, and moisturizers. Additives may be from natural extracts, or may be synthetic, or a combination of both. Flavorings and additives may include, for example, aloe, eucalyptus oil, and menthol. Additives may also include pharmaceutical, medical, or nutritional compositions such as, for example, antibiotics and steroids.

### Sinus and Nasal Conditions

Nasal and sinus conditions that may be treated by the current formulation include, for example, sinusitis, chronic sinusitis, dryness, atrophic rhinitis, inflammation, catarrh, rhinitis, allergic rhinitis, common cold, and postnasal drip.

In some embodiments, the nasal wash may be used to treat a condition that is not manifested in the nasal or sinus cavities, for example, cough, sore throat, or stuffy ears.

### Isotonic

The current composition may provide for an isotonic solution. An isotonic solution may have a solute concentration that is the same or similar to the solute concentration as a mammalian cell or a bodily fluid, for example blood. An isotonic solution may be a solution that does not promote the net movement of water.

In physiological systems, for example, an isotonic solution may not cause a net movement of water into or out of a cell. A hypertonic solution may cause a net movement of water out of a cell, while a hypotonic solution may cause a net movement into a cell. Tonicity is affected by the amount of solutes in a solution, especially solutes that cannot easily cross a cell membrane. Generally, in biological systems, tonicity is mostly affected by the salt concentration of the solution, but other molecules and compounds may also contribute to tonicity.

A salt is an ionic compound composed of cations and anions, which may dissociate in water to produce separate positively and negatively charged ions. The tendency of a salt to dissociate is a function of the salt's dissociation constant. Dissociation constants reflect the strength of the interaction between the positively and negatively charged ions, and the tendency of the ions to dissociate in solution.

The salt of the current composition may be sodium chloride. An isotonic solution of sodium chloride may be approximately 0.9% sodium chloride, or 0.9 g (grams) NaCl in 100 ml of H₂O. 100 ml of water may weigh about 100 g. In other embodiments the solution may be between 1.0 and 0.8% sodium chloride. 0.9% sodium chloride solutions are generally isotonic with most mammalian cells, but solutions with less or more than 0.9% sodium chloride may also be isotonic.

In some embodiments the salt solution may contain additional chloride salts such as potassium chloride, magnesium chloride, calcium chloride, and combinations thereof. In further embodiments, non-chloride salts may also be present. In still further embodiments the formulation may include phosphate, sulfate, or carbonate salts.

Embodiments comprised of salts in addition to, or other than, NaCl may be greater than or less than a 0.9% solution. Compositions that further include additives that may alter the tonicity of the nasal wash solution may comprise a reduced amount of salt or buffer or both.

### pH Buffers

pH is a measure of the acidity or basicity of a solution and is written as a log of the hydrogen ions in solution. Acidic solutions have a pH less than 7, while basic solutions have a pH above 7. A pH of 7.0 is said to be neutral. Pure water has a pH of about 7.0. The pH of blood may be slightly basic, about 7.4.

pH may be measured electrically or by the use of color changing solutions or compounds. In some cases a colormetric solution may be added dropwise to a test solution, or a test solution may be applied to various papers containing a colormetric compound/solution (test strips). pH may also be assayed electronically by pH meter and comparison to solutions of known pH. pH values may vary +/−0.1. Thus, a pH of 7.6 may include a range from about 7.5 to about 7.7.

pH buffers may aid in maintaining the pH of a solution at a pre-determined range even when the amount of hydrogen atoms change. Hydrogen ion concentrations may change in a solution due to introduction of an acid or a base, or the hydrogen ion concentrations may increase or decrease in response to changes in temperature. In biological systems, sodium bicarbonate may help keep the pH of a given solution near a physiological reading of 7.4. Sodium bicarbonate may also help to resist rapid pH changes in an acidic direction.

The present formulation may include sodium carbonate and sodium ascorbate. Sodium carbonate has the molecular formula NaHCO₃, and a molecular weight of approximately 84 g/mole. Sodium ascorbate has the chemical formula, C₆H₇NaO₆, and a molecular weight of approximately 198 g/mole. Sodium ascorbate may produce an acidic solution, which may aid in inhibiting bacterial growth. Further, sodium ascorbate may react with hydrochloric acid, to create ascorbic acid.

In various embodiments the composition may include other buffers to achieve the desired results. For example, in some embodiments sodium phosphate dibasic, Na₂HPO₄, may be mixed with sodium phosphate monobasic, NaH₂PO₄, to prepare a composition that, when mixed with water, may result in a pH around 6.7. In other embodiments, sodium phosphate monobasic and/or dibasic may be mixed with sodium bicarbonate and/or sodium ascorbate to create a composition that may result in a solution of the desired pH.

### Dose

In some embodiments the dose may be an individually packaged amount of the composition. In other embodiments the dose may be designated by volume and measured with a device such as a measuring spoon, dropper, or cup. In further embodiments the dose may also be designated by a specific volume or weight, as in a specified number of milliliters or grams. Doses are intended to be added to a specified volume of water to produce a generally isotonic composition.

In embodiments where the formulation is provided in bulk, the formulation may include a device for measuring the appropriate amount of composition for a specified volume of water. In other embodiments, the formulation may be provided in liquid form to be diluted by a specified factor, such as for example, a 10×, 20×, 50×, or 100× solutions. As described above, varying the amount of formulation added to the appropriate volume of water will create other than an isotonic solution.

In some embodiments, the composition may be provided as a solution. In some embodiments, the composition may be provided as a solution for use as a nasal spray. Nasal sprays or mists may help to treat nasal conditions, for example, dryness. Nasal sprays may generally introduce less volume of a solution into the nasal and sinus cavities than a wash or lavage application.

### Nasal Sinus Environment

As used herein, the terms nasal and sinus are interchangeable, as in nasal cavity and sinus cavity. These terms are used herein to refer to both the nasal cavity and the paranasal sinus cavities that communicate with the nasal cavity.

It will be apparent to those of ordinary skill in the art that variations and alternative embodiments may be made given the foregoing description. Such variations and alternative embodiments are accordingly considered within the scope of the present invention.

### Example One

### Growth Inhibition of Staphylococcus

Four solutions were tested for their ability to support growth of S. epidermidis. The solutions were: Solution A, 1 packet of the present formulation dissolved in 240 mL sterile dH₂O; Solution B, 1 packet of NeilMed Sinus Rinse dissolved in 240 mL sterile dH₂O; Solution C, 1 packet of Breathe Ease XL dissolved in 500 mL sterile dH₂O. The final solution, Solution D, PBS (Phosphate Buffered Saline, pH 7.4), was included as a control.

A stock culture of S. epidermidis were plated a blood agar (BA) plate and incubated overnight at 36±2° C. Individual colonies are selected and grown in peptone.

Overnight cultures were adjusted to a turbidity equal to that of a 0.5 McFarland standard, and assumed to contain 1.5×10⁸ CFU (colony forming units)/mL. These cultures were diluted 1:10 in peptone to obtain a concentration of 1.5×10⁷CFU/mL.

On Day 0, 200 μL of the 1.5×10⁷CFU/mL organism-suspension was diluted into 20 mL of the selected test solution (A, B, or C) and the control solution (D) to obtain a concentration of 1.5×10⁵CFU/mL. This inoculated solution was then immediately diluted 1:10 and 50 μL plated onto the appropriate agar plate for overnight growth at 36±2° C. This was the O-hour sample. Inoculated test solutions were incubated at 36±2° C. for the duration of the experiment.

At 8, 24, 32, 50, and 74-hour intervals, each inoculated test solution was further diluted 1:10 and 1:1000, a 50 μL aliquot removed, and each aliquot spiral plated on the appropriate agar plate. All plates were incubated at 36±2° C. overnight, and then removed from incubation for counting and recording the number of colonies on each plate.

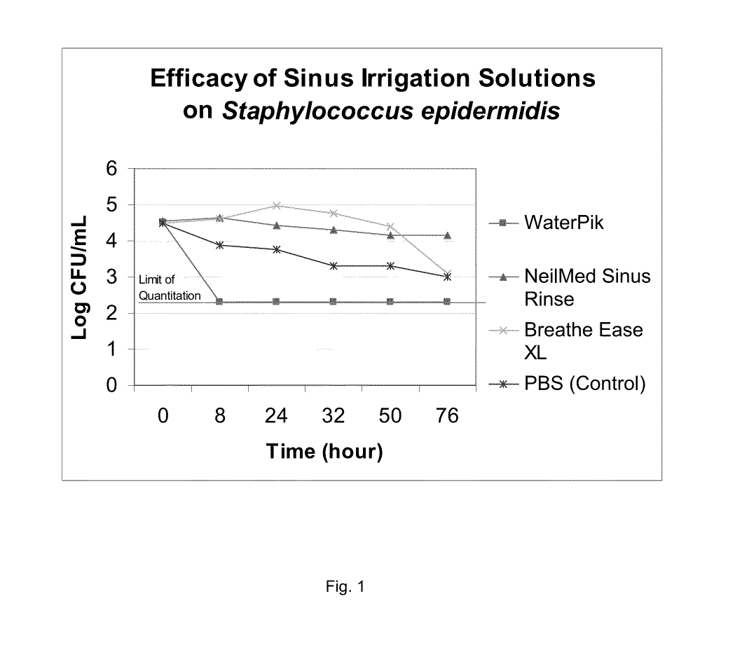

The following tables are presented graphically in FIG. 1. Table I presents the raw data and Table II presents the data of Table 1 in log base 10. These results show that the present formulation does not support growth of staphylococcus bacteria and reduces the number of bacterial cells below detectable levels in less than 8 hours.

TABLE I

CFU/mL

| | Presently | NeilMed | | |
|---|---|---|---|---|
| Time | Disclosed | Sinus | Breathe | PBS |
| (Hrs.) | Formulation | Rinse | Ease XL | (Control) |
| 0 | 3.2E+04 | 3.5E+04 | 3.1E+04 | 3.1E+04 |
| 8 | 2.0E+02 | 4.4E+04 | 4.1E+04 | 7.8E+03 |
| 24 | 2.0E+02 | 2.6E+04 | 9.2E+04 | 5.6E+03 |
| 32 | 2.0E+02 | 2.0E+04 | 5.9E+04 | 2.0E+03 |
| 50 | 2.0E+02 | 1.4E+04 | 2.5E+04 | 2.0E+03 |
| 76 | 2.0E+02 | 1.4E+04 | 1.2E+03 | 1.0E+03 |

TABLE II

LOG₁₀ CFU/mL

| | Presently | NeilMed | | |
|---|---|---|---|---|
| Time | Disclosed | Sinus | Breathe | PBS |
| (Hrs.) | Formulation | Rinse | Ease XL | (Control) |
| 0 | 4.51 | 4.54 | 4.49 | 4.49 |
| 8 | 2.30 | 4.64 | 4.61 | 3.89 |
| 24 | 2.30 | 4.41 | 4.96 | 3.75 |
| 32 | 2.30 | 4.30 | 4.77 | 3.30 |
| 50 | 2.30 | 4.15 | 4.40 | 3.30 |
| 76 | 2.30 | 4.15 | 3.08 | 3.00 |

What is claimed is:

1. A nasal wash composition comprising
a salt; and
a pH buffer, wherein
when the salt and the pH buffer are mixed with a prescribed volume of water an isotonic solution of pH below 7.0 is produced.

2. The composition according to claim 1, wherein the pH of the solution is 6.7.

3. The composition according to claim 1, wherein the pH buffer is selected from sodium ascorbate and sodium bicarbonate.

4. The composition according to claim 1, further comprising one or more additives selected from the group consisting of moisturizers, flavorants, oils, herbs, and medicaments.

5. The composition according to claim 4, wherein the additive is selected from the group consisting of eucalyptus oil, menthol, and aloe.

6. The composition according to claim 1, comprising
sodium chloride between 80% to 88% by weight of the composition;
sodium ascorbate between 9% to 20% by weight of the composition; and
sodium bicarbonate between 0% to 2% by weight of the composition.

7. The composition according to claim 1, wherein the prescribed amount of water is approximately 100-times the weight of the composition.

8. The composition according to claim 1, wherein the composition is dry.

9. The composition according to claim 1, wherein the composition is packaged in pre-measured amounts or provided in bulk form.

10. The composition according to claim 1 for use in a nasal spray.

11. A method of preparing a nasal wash solution comprising:
combining
a pre-determined amount of a salt, and
a pre-determined amount of a pH buffer, and
a pre-determined volume of water,
to create an isotonic solution, wherein said solutions has a pH of below 7.0, thus preparing a nasal wash solution.

12. The method of claim 11, wherein the salt and the pH buffer are combined into a composition.

13. The method of claim 12, wherein the composition further comprises one or more additives selected from the group consisting of moisturizers, flavorants, oils, herbs, and medicaments.

14. The method of claim 13, wherein the additive is selected from the group consisting of eucalyptus oil, menthol, and aloe.

15. The method of claim 11, wherein the pH of the isotonic solution is between 6.6 and 6.8.

16. The method of claim 15, wherein the pH of the solution is 6.7.

17. The method of claim 11, wherein the salt is selected from one or more of sodium ascorbate and sodium chloride.

18. The method of claim 11, wherein the pH buffer is selected from one or more of sodium ascorbate and sodium bicarbonate.

19. The method of claim 12, wherein the composition comprises
sodium chloride between 80% to 88% by weight of the composition;
sodium ascorbate between 9% to 20% by weight of the composition; and
sodium bicarbonate between 0% to 2% by weight of the composition.

20. The method of claim 11, wherein the water is one or both of distilled or de ionized.

21. A method of treating a nasal or sinus condition comprising
combining an amount of nasal composition with a volume of water to create a salt solution with a pH below 7.0, wherein the ratio of composition to water is about 1:100; and
introducing the isotonic salt solution into the sinus or nasal cavities of a patient in need of such treatment.

22. The method according to claim 21, wherein the solution is selected from isotonic, hypertonic, and hypotonic.

23. The method according to claim 21, wherein the solution inhibits bacterial growth.
